(19) United States
(12) Plant Patent Application Publication
(10) Pub. No.: US 2024/0008379 P1
(43) Pub. Date: Jan. 4, 2024

(54) CANNABIS PLANT NAMED '21AF0716'

(50) ;

(71) Applicant: Grace Executive Service, Inc., Oakland, CA (US)

(72) Inventor: Caitlin Watt, Oakland, CA (US)

(21) Appl. No.: 18/445,188

(22) Filed: May 16, 2023

Related U.S. Application Data
(60) Provisional application No. 63/365,117, filed on May 20, 2022.

(51) Int. Cl. A01H 6/28

(52) U.S. Cl. PLT/258

(57) ABSTRACT

The unique annual herbaceous Cannabis plant variety ‘21AF0716’ is provided. The variety can be distinguished by its outstanding features of increased resistance to powdery mildew, higher total yield, and larger inflorescences compared to its parents.

### CROSS REFERENCE TO RELATED APPLICATIONS

The present Application for Patent claims benefit of Provisional Application No. 63/365,117 entitled “CANNABIS PLANT NAMED ‘21AF0716” filed May 20, 2022, which is hereby expressly incorporated by reference herein.

Latin name of the genus and species: Genus—Cannabis. Species—sativa.

Variety denomination: The new Cannabis plant claimed is of the variety denominated ‘21AF0716’.

### BACKGROUND OF THE INVENTION

### Field of the Invention

The present invention relates to a new and distinct annual variety of C. sativa, which has been given the variety denomination of ‘21AF0716’. ‘21AF0716’ is intended for use as a plant both commercially as well as for home growers, including as part of a breeding program to develop improved Cannabis varieties.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments and is also the active ingredient in the FDA-approved drug Epidiolex®. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1;20(11):2083-2085; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Non-hemp forms of Cannabis, frequently referred to as marijuana, have been legalized for medicinal use in many states and for recreational use (sometimes called “adult use”) in a growing number of states. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘21AF0716’.

The new C. sativa variety is a selection resulting from a sexual cross of C. sativa plants in San Mateo County, California, in September 2021, involving a seed parent known as ‘Gelato 33’ (unpatented) and a pollen parent known as ‘Purple Punch’ (unpatented).

Selection criteria were high yield, dense flowers, and resistance to powdery mildew under greenhouse conditions.

The selection was subsequently evaluated for a number of years in California.

Asexual reproduction of the new variety by cutting propagation since 2021 in San Mateo County, California has demonstrated that the new variety reproduces true to type with all of the morphological characteristics, as herein described, firmly fixed and retained through successive generations of such asexual propagation.

Certain characteristics of the new variety have been repeatedly observed and can be used to distinguish ‘21AF0716’ as a new and distinct variety of C. sativa plant. Plants of the new variety differ from plants of the seed parent ‘Gelato 33’ primarily in node spacing between inflorescences, increased inflorescence size, inflorescence density, yield as total weight per plant, and floral aroma. Plants of the new variety differ from plants of the pollen parent ‘Purple Punch’ in increased continued growth under flowering light, increased inflorescence size, yield as total weight per plant, and floral aroma. The variety expresses increased resistance to powdery mildew, higher total yield, and larger inflorescences than either parent. It exhibits continued growth under flowering light and dark purple coloration on the inflorescences similar to the seed parent and exhibits the inflorescence density and resistance to production of stress-induced male flowers of the pollen parent.

### BRIEF DESCRIPTION OF THE DRAWINGS

The accompanying photographic illustrations show the typical appearance of the new variety ‘21AF0716’. The colors are as nearly true as is reasonably possible in a color representation of this type. Colors in the photographs may differ slightly from the color values cited in the detailed botanical description which accurately describes the colors of the new plant.

FIG. 1 is a photograph of the new variety ‘21AF0716’, demonstrating the finished flower on a row of mature plants (2 months of flowering under greenhouse conditions).

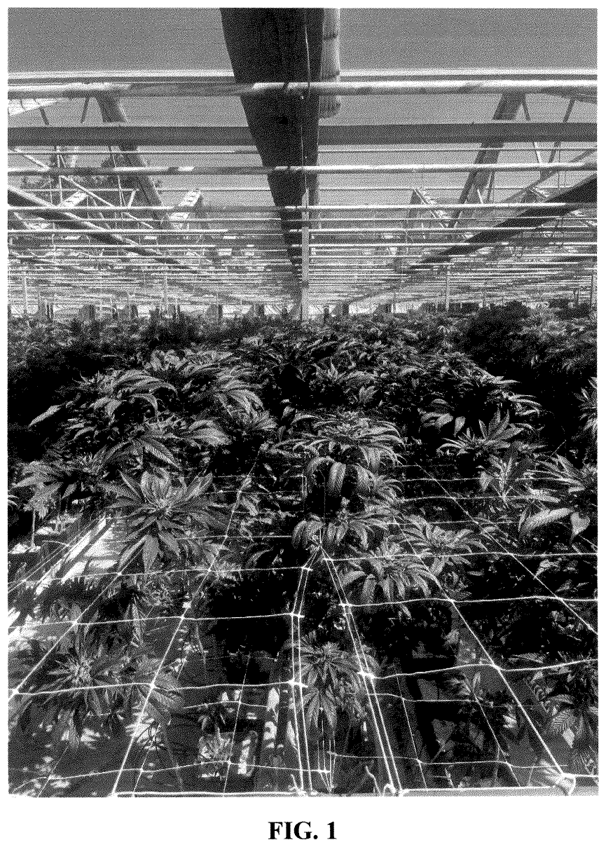

FIG. 2 is a photograph of the mature flowers of the new variety ‘21AF0716’.

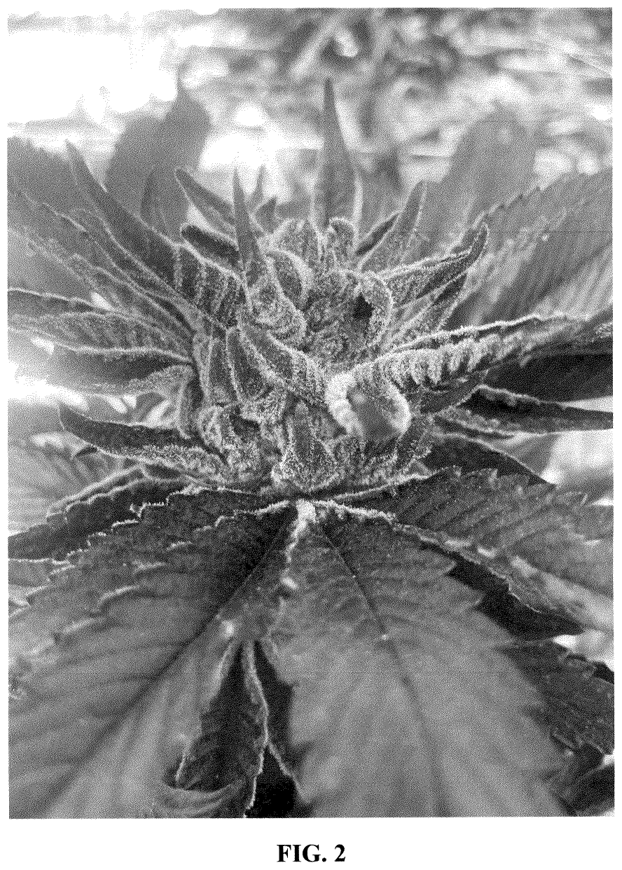

FIG. 3 is a photograph of young flowers of new variety ‘21AF0716’ at 1 month into flowering under greenhouse conditions.

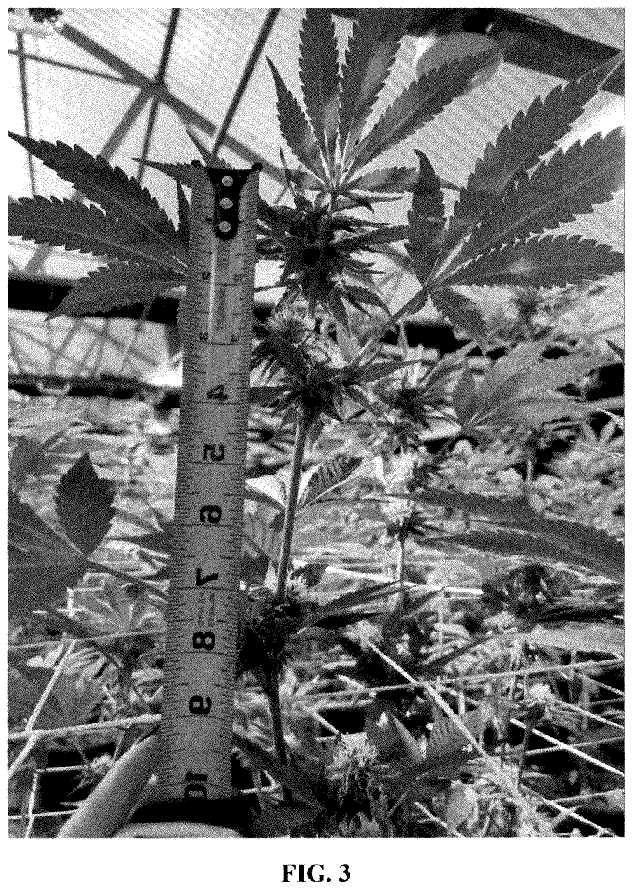

FIG. 4 is a photograph of young flowers of the new variety ‘21AF0716’ at 5 weeks into flowering under greenhouse conditions.

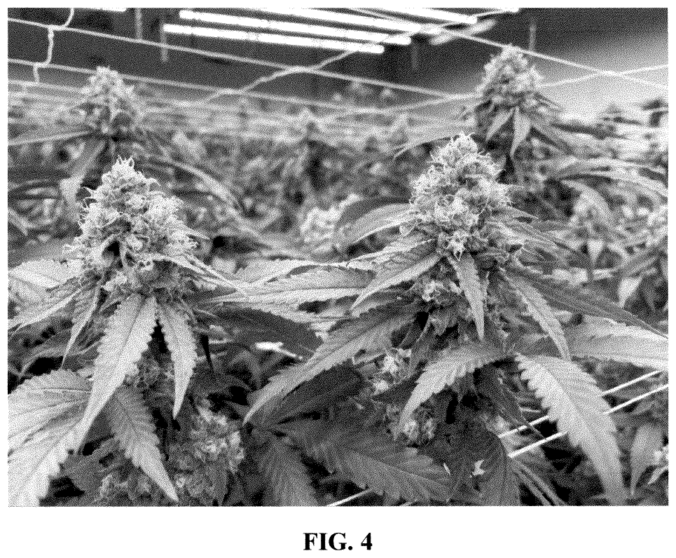

FIG. 5 is a photograph of the immature plant of the new variety ‘21AF0716’ at 3 months of growth under vegetative conditions.

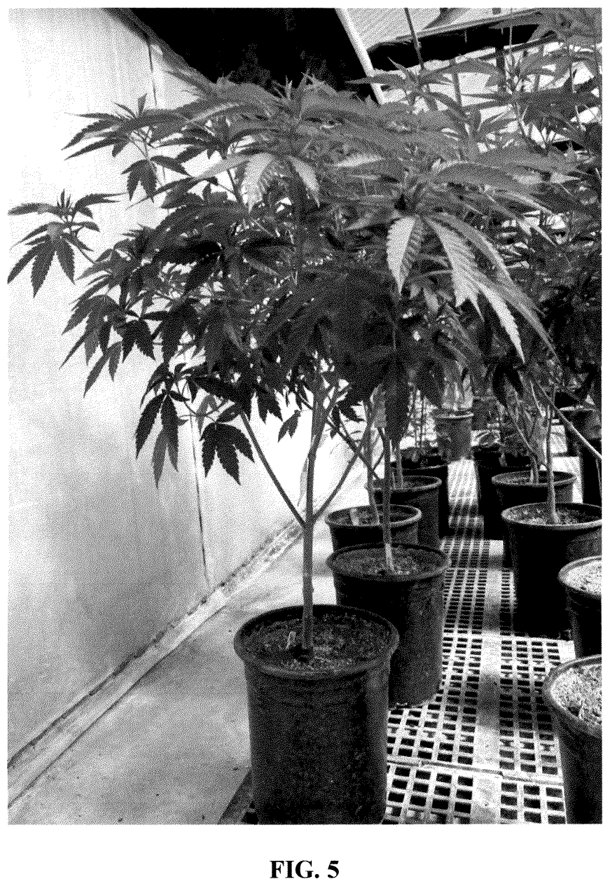

### DETAILED BOTANICAL DESCRIPTION

The following detailed description sets forth the distinctive characteristics of ‘21AF0716’. The datum which defines these characteristics was collected from asexual reproductions of the original selection. Dimensions, sizes, colors, and other characteristics are approximations and averages set forth as accurately as possible. The plant history was taken on mother plants approximately 3 months old, and the descriptions relate to plants grown in Half Moon Bay, California.
Type: Herbaceous annual.
Classification:
   a. Family.—Cannabaceae.
   b. Genus.—Cannabis.
   c. Species.—sativa.
   d. Common name.—Marijuana.
Parentage: ‘Gelato 33’ x ‘Purple Punch’.

### PLANT

General:
   a. Parentage—i. Female parent— ‘Gelato 33’. ii. Reversed Female parent— ‘Purple Punch’.
   b. Growth habit.13 Medium growth habit with strong branches and compact structure.
   c. Hardiness.—USDA Zone 2 and higher.
   d. Average height.—56 inches.
   e. Average spread.—28 inches.
   f. Productivity.—623 grams.
   g. Disease resistance/susceptibility.—Low disease susceptibility under standard growing conditions.
   h. Pest resistance/susceptibility.—Higher resistance to powdery mildew than either of its parents.
   i. Fragrance.—Cake, cookie and gassy aroma.
   j. Proportion of hermaphrodite plants.—Low.
   k. Proportion of female plants.—Female plants are agronomically significant and seeds are feminized.
   l. Type of inflorescence.—High branched compound racemose.
   m. Photoperiodicity.—Short day photoperiod sensitive.
   n. Pollen.—i. Amount — Scarce. ii. Color —White.

### STEM/CENTRAL STALK

General:
   a. Average diameter of stem.—1 inch.
   b. Average length of stem.—48 inches.
   c. Shape.—Tubular.
   d. Depth of grooves.—Shallow.
   e. Pith in cross section.—Medium.
   f. Trichome type.—Few trichomes.
   g. Color.—Green.

### LEAF

General:
   a. Leaf arrangement.—Alternate.
   b. Leaf shape.—Palmately Compound.
   c. Leaf margins.—Serrate.
   d. Leaf apex shape.—Acuminate.
   e. Leaf base shape.—Attenuate.
   f. Number of leaflets per leaf.—5-7.
   g. Trichome type (top surface).—On sugar leaves near inflorescences numerous cystolithic hair, bulbous, capitate sessile or capitate stalked.
   h. Texture of top and bottom surfaces.—Pubescent leaves.
   i. Venation type.—Pinate.

### PETIOLE

General:
   a. Surface texture.—Pubescent.
   b. Anthocyanin intensity.—Intense at end of growing season.
   c. Color.—Green to purple.

### FEMALE FLOWER

General:
a. Natural flowering season.—Summer.
b. Color—Purple. i. Trichome type — Numerous cystolithic hair, bulbous, capitate sessile or capitate stalked, trichomes start clear turning to amber over time. ii. Color — Green to purple.

‘21AF0716’ can include a cannabinoid profile as set forth in Table 1. Chemotypic characteristics of the new variety are variable based upon cultivation conditions, as is typical of Cannabis plants. Accordingly, while chemotypic information provided herein is representative of performance of the new varieties under a particular set of cultivation conditions, it is not limiting on other chemotypic profiles obtainable under a different set of cultivation conditions. Thus, the values set forth in Table 1 do not reflect the only possible range of outcomes that can be obtained from plants of the new variety. These values are merely exemplary of observed values (middle column) and predicted normal variations from the observed values.

TABLE 1
Exemplary Profiles of Key Cannabinoids.

| Cannabinoid | Percent | Percent | Percent | Percent | Percent |
|---|---|---|---|---|---|
| THCa | 17.63625 | 18.812 | 23.515 | 28.218 | 29.39375 |
| Delta 9 THC | 0.472425 | 0.50392 | 0.6299 | 0.75588 | 0.787375 |
| CBGa | 0.4206 | 0.44864 | 0.5608 | 0.67296 | 0.701 |
| CBCa | 0.141375 | 0.1508 | 0.1885 | 0.2262 | 0.235625 |
| THCVa | 0.054075 | 0.05768 | 0.0721 | 0.08652 | 0.090125 |
| CBDa | 0.03255 | 0.03472 | 0.0434 | 0.05208 | 0.05425 |
| CBG | 0.031725 | 0.03384 | 0.0423 | 0.05076 | 0.052875 |
| Total THC | 15.939375 | 17.002 | 21.2525 | 25.503 | 26.56563 |
| Total CBD | 0.028575 | 0.03048 | 0.0381 | 0.04572 | 0.047625 |
| Total | 11.0266 | 17.64256 | 22.0532 | 26.46384 | 27.5665 |
| Cannabinoid | | | | | |

*Total THC = (THCA * 0.877) + THC (i.e., delta 9 THC) + delta 8 THC
**Total CBD = CBD + (CBDa * 0.877)
***Total Cannabinoids = Total THC + Total CBD + Total CBG + Total THCV + Total CBC + Total CBDV + Δ8THC + CBL + CBN]

1. A new and distinct variety of Cannabis plant named ‘21AF0716’, as illustrated and described herein.
